(19) United States
(12) Patent Application Publication
(10) Pub. No.: US 2011/0236919 A1
(43) Pub. Date: Sep. 29, 2011

(54) PROCESS FOR RESTRICTING CARBON MONOXIDE DISSOLUTION IN A SYNGAS FERMENTATION

(76) Inventors: James Allen Zahn, Campton Hills, IL (US); Seth William Fischbein, Lombard, IL (US)

(21) Appl. No.: 12/730,763

(22) Filed: Mar. 24, 2010

(51) Int. Cl. C12Q 1/32; C12Q 1/06; C12P 7/16; C12P 7/06

(52) U.S. Cl. 435/26; 435/39; 435/160; 435/161

(57) ABSTRACT

A process to temporarily and selectively control the dissolved concentration of CO in the feed medium portion of a fermentation liquid by addition of a surface tension active compound (STAC), particularly during seed train scale-up operations and during recovery from process upsets, to support improved production of biofuels from a syngas fermentation. The process inoculates the fermentation liquid when the cell density level is from 0.05 to 0.5 OD with the surface tension active compound at a concentration of from 20 to 800 mg per liter of syngas fermentation liquid. The surface tension active compound may be a polyol or glycol.

### FIELD OF THE INVENTION

This invention relates to a process for biological conversion of CO, H₂, and mixtures comprising CO and/or H₂ to biofuel products.

### BACKGROUND

Biofuels production for use as liquid motor fuels or for blending with conventional gasoline or diesel motor fuels is increasing worldwide. Such biofuels include, for example, ethanol and n-butanol. One of the major drivers for biofuels is their derivation from renewable resources by fermentation and bioprocess technology. Conventionally, biofuels are made from readily fermentable carbohydrates such as sugars and starches. For example, the two primary agricultural crops that are used for conventional bioethanol production are sugarcane (Brazil and other tropical countries) and corn or maize (U.S. and other temperate countries). The availability of agricultural feedstocks that provide readily fermentable carbohydrates is limited because of competition with food and feed production, arable land usage, water availability, and other factors. Consequently, lignocellulosic feedstocks such as forest residues, trees from plantations, straws, grasses and other agricultural residues may become viable feedstocks for biofuel production. However, lignocellulosic materials are inherently recalcitrant to conversion.

A promising technology path for conversion of lignocellulosic feedstock to biofuel is to convert lignocellulosic biomass to syngas (also known as synthesis gas, primarily a mix of CO, H₂ and CO₂ with other components such as CH₄, N₂, NH₃, H₂S and other trace gases) and then ferment this gas with anaerobic microorganisms to produce biofuels such as ethanol or n-butanol or chemicals such as acetic acid, butyric acid and the like. This path is inherently efficient because the gasification step converts all of the components to syngas with good efficiency (i.e., greater than 75%), and some strains of anaerobic microorganisms can convert syngas to ethanol, n-butanol or other chemicals with high (i.e., theoretically greater than 90%) efficiency. Moreover, syngas can be made from many other carbonaceous feedstocks such as natural gas, reformed natural gas, peat, petroleum coke, coal, solid waste and land fill gas, making this a more universal technology path. A complete description of the syngas to biofuel process is provided in U.S. Pub. Nos. 20090215163, 20080305539, 20080305540, 20090035848, 20090215139, 20090017514, 20090215153, and 20090105676, which are hereby incorporated by reference in their entirety.

The efficiency of the syngas to biofuel technology pathway in terms of output of biofuels is largely dependent upon hydrogenase activity of the anaerobic microorganisms, particularly since it is likely that the health of the microorganisms is related to their hydrogen uptake. All other factors remaining constant, if hydrogenase activity increases, output in L/day increases, and if hydrogenase activity is impaired for any reason, output decreases. This takes place in part because of the stoichiometric ratios required for the above-described process to operate:

6CO+3H2→C2H5OH+4CO2

6H2+2CO2→C2H5OH+3H2O

However, the hydrogenase activity of many strains of the anaerobic microorganisms that convert syngas to biofuels is highly sensitive to dissolved carbon monoxide levels in the fermentation liquid, the medium contained in the vessel or fermentor which comprises a feed broth and suitable microorganisms

High dissolved concentrations of CO are routinely observed during seed culture scale-up activities, during standard operation of the process, and during recovery from process upsets. High CO gas concentrations may result from plasma gasification and the high hydrostatic pressure associated with certain preferred reactors. Such high concentrations result in the inhibition of cell growth and low gas conversion efficiencies for hydrogen.

Typically, the control of carbon monoxide solubility in the fermentation liquid is managed through three separate methods: (1) limiting the gas flow to the reactor so that the uptake of the gas by the microorganisms in the fermentation liquid is faster than the dissolution rate into the feed medium of the fermentation liquid; (2) dilution of the gas that enters the vessel or reactor with an inert gas, such as nitrogen, so that the gas feed stream contains a significantly lower concentration of CO; or (3) reduction of the intensity of the gas-liquid contact through the reduction of agitation and/or through the reduction of liquid or gas flow to the gas reductor nozzles.

Often during initial culture of the microorganisms or after a process upset, a combination of all three of the CO control measures is necessary due to the low metabolic activity and/or low cell density of the bacteria. Unfortunately, use of these control mechanisms significantly limits total gas conversion efficiency.

### SUMMARY OF THE INVENTION

The present invention provides a process to temporarily and selectively control the dissolved concentration of CO in the feed medium portion of the fermentation liquid by addition of a surface tension active compound (STAC), particularly during seed train scale-up operations and during recovery from process upsets, to support improved production of biofuels from a syngas fermentation.

More specifically, the present invention provides a process for restricting CO dissolution into a syngas fermentation liquid in a vessel during the feed phases of production of biofuels where at least 2 mg of CO per liter of syngas fermentation liquid are present, said process comprising the steps of: passing a feed gas stream comprising CO, H2, and CO2 through the syngas fermentation liquid; monitoring the cell density level of the syngas fermentation liquid; and when the cell density level is from 0.05 to 0.5 OD, inoculating the fermentation liquid with a STAC at a concentration of from 20 to 800 mg per liter of syngas fermentation liquid.

In another embodiment, the present invention provides a process for restricting CO dissolution into a syngas fermentation liquid in a vessel during the feed phases of production of biofuels where at least 2 mg of carbon monoxide per liter of syngas fermentation liquid are present, said process comprising the steps of: passing a feed gas stream comprising CO, H2, and CO2 through the fermentation liquid; monitoring the level of hydrogenase activity of cells in the fermentation liquid, either via measurement of H2 uptake in mmol/L/hour or via measurement of H2 conversion as a percentage of mass; and inoculating the broth fraction of the syngas fermentation liquid with from 20 to 800 mg per liter of broth of a STAC when either the H2 uptake level decreases by thirty (30) percent or more over a 48 hour period or the H2 conversion as a percent of mass is from zero to twenty-five (25) percent.

Further, the present invention provides a process for restricting CO dissolution into a syngas fermentation liquid in a vessel during recovery from a process upset where at least 2 mg of CO per liter of fermentation liquid are present, said process comprising the steps of: passing a feed gas stream comprising CO, H2, and CO2 through the fermentation liquid; monitoring the level of hydrogenase activity of the cells in the fermentation liquid, either via measurement of H2 uptake in mmol/L/hour or via measurement of H2 conversion as a percentage of mass; and inoculating the broth fraction of the syngas fermentation liquid with from 10 to 400 mg per liter of broth per day of a STAC when either the H2 uptake level decreases by thirty (30) percent or more over a 48 hour period or the H2 conversion as a percent of mass is from zero to twenty-five (25) percent.

### BRIEF DESCRIPTION OF THE DRAWINGS

FIG. 1 shows the influence of H₂ uptake on ethanol productivity by an ethanologenic clostridium utilizing syngas to produce ethanol in a stirred tank reactor.

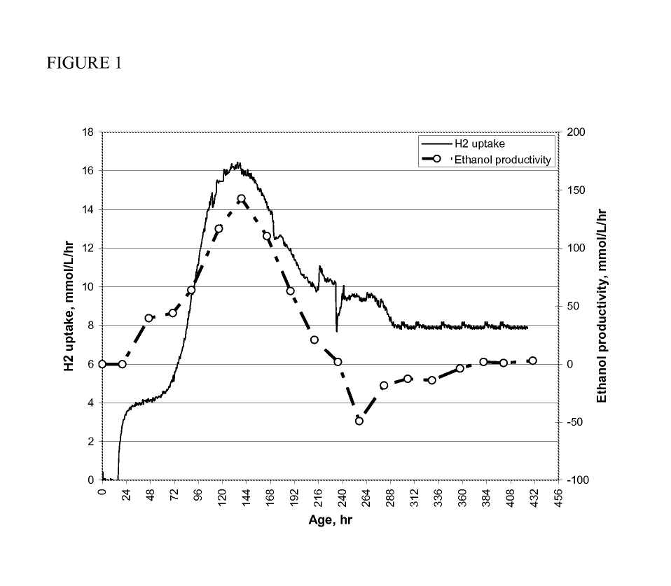

FIG. 2 shows the effect of dissolved CO in mg/L at saturation on hydrogen uptake in mmol H₂/g DCW/hr in Clostridium ragsdalei.

FIG. 3 shows a measurement of the dynamic surface tension of fermentation liquid containing C. autoethanogenum strain 3037 containing a cell density of 6.3 g DCW per liter of feed broth. The blank contained centrifuged cells with no added ethanol, and the control contained cells with 30 g/L of ethanol and 10 g/L of acetate with a pH of 6.2 surface tension active compound was added to a final concentration of 50 mg/L.

FIG. 4 shows the effect of pulse additions of the STAC Cognis FBA 975US on H2 uptake, CO uptake, and CO₂ uptake by Clostridium autoethanogenum, which was grown in a 2 L continuous stirred tank reactor.

FIG. 5 shows the effect of pulse additions of the STAC Cognis FBA 975US on H₂ conversion and CO conversion by Clostridium autoethanogenum, which was grown in a 2 L continuous stirred tank reactor.

### DETAILED DESCRIPTION OF THE INVENTION

The present invention details a method of ameliorating the problems associated with the dissolution of high concentrations of CO in fermentation liquid during the syngas to biofuels process. High levels of CO can adversely affect cell growth, overall efficiency of the syngas to biofuel conversion process, and, ultimately, volume of output of biofuel. In particular, concentrations of CO greater than 2 mg per liter of fermentation liquid cause such adverse effects during the seed train scale-up phases of production and during recovery phases from any process upset that might occur.

Suitable microorganisms for use in the syngas fermentation liquid generally live and grow under anaerobic conditions, meaning that dissolved oxygen is essentially absent from the fermentation liquid. Any suitable microorganisms that have the ability to convert the syngas components: CO, H₂, CO₂ individually or in combination with each other or with other components that are typically present in syngas may be utilized. Suitable microorganisms and/or growth conditions may include those disclosed in U.S. patent application Ser. No. 11/441,392, which discloses a biologically pure culture of the microorganism Clostridium carboxidivorans having all of the identifying characteristics of ATCC No. BAA-624; and U.S. patent application Ser. No. 11/514,385, which discloses a biologically pure culture of the microorganism Clostridium ragsdalei having all of the identifying characteristics of ATCC No. BAA-622; both of which are incorporated herein by reference in their entirety. Clostridium carboxidivorans may be used, for example, to ferment syngas to ethanol and/or n-butanol. Clostridium ragsdalei may be used, for example, to ferment syngas to ethanol.

Suitable microorganisms and growth conditions include the anaerobic bacteria Butyribacterium methylotrophicum, having the identifying characteristics of ATCC 33266 which can be adapted to CO and used and this will enable the production of n-butanol as well as butyric acid as taught in the references: “Evidence for Production of n-Butanol from Carbon Monoxide by Butyribacterium methylotrophicum,” Journal of Fermentation and Bioengineering, vol. 72, 1991, p. 58-60; “Production of butanol and ethanol from synthesis gas via fermentation,” FUEL, vol. 70, May 1991, p. 615-619. Other suitable microorganisms include Clostridium ljungdahlii, with strains having the identifying characteristics of ATCC 49587 (U.S. Pat. No. 5,173,429) and ATCC 55988 and 55989 (U.S. Pat. No. 6,136,577) that will enable the production of ethanol as well as acetic acid and Clostridium autoethanogenum, an anaerobic bacterium with the characteristics of DSMZ 10061, that produces ethanol from carbon monoxide. Jamal Abrini, Henry Naveau, Edomond-Jacques Nyns, Arch Microbiol., 1994, 345-351; Archives of Microbiology 1994, 161: 345-351. All of these references are incorporated herein in their entirety. Finally, Clostridium coskatii can be utilized, with strains having the identifying characteristics of ATCC PTA-10522, to convert syngas to ethanol.

During the seed train scale-up phases of production, the microorganisms in the fermentation liquid are cultured in a feed medium which may comprise portions of mineral stock solution, trace metal stock solution, vitamin stock solution, cysteine stock solution, Cognis Clerol FBA975 stock solution or a commercially available equivalent, and water. A preferred preparation of the feed medium is described in TABLE 1. Additionally, U.S. Pub. No. 20080057554 A1, the contents of which are hereby incorporated by reference, further discloses the conditions and contents of suitable fermentation liquid for bioconversion CO and H₂/CO₂ using anaerobic microorganisms.

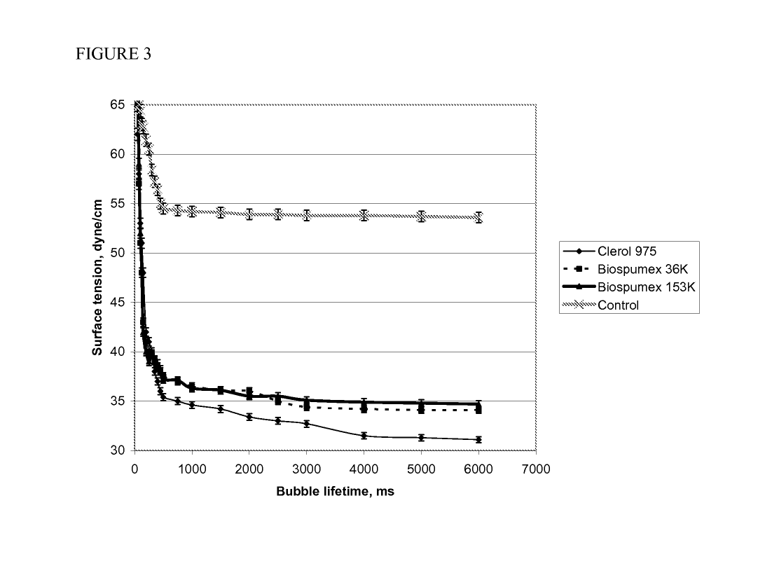

A feed gas stream consisting of syngas which comprises CO, H₂, and CO₂ is passed through the fermentation liquid. The fermentation liquid is then gradually passaged to larger vessels as the log phase of growth is reached using standard passaging technique well known to those of ordinary skill in the art. The process may use any vessel that can contain the fermentation liquid and provide any desired environment for the process, but will typically comprise a bioreactor that retains the microorganisms suspended in the fermentation liquid. Preferred vessels include a bubble column bioreactor, a continuous stirred tank bioreactor (CSTR), and a two-stage bioreactor. Such bioreactors often contain large volumes of fermentation liquid that require liquid depth of 20 meters or more. Particularly during these stages, it is imperative that the dissolved concentrations of CO be limited in order to prevent inhibition of cell growth and low gas conversion efficiencies for hydrogen. It has been found that the addition of a STAC, in a preferred embodiment a polyol or glycol STAC, will significantly decrease the surface tension of gas bubbles, causing efficient bubble collapse and coalescence of bubbles into larger bubbles. See FIG. 3, which shows the rapid decrease in surface tension over time after inoculation with a STAC to a concentration of 50 mg/L.

Additionally, use of STACs are well-suited for initial phases of fermentation scale-up because the volume of a given vessel or reactor following inoculation with fermentation liquid is typically 20% of the normal operating volume, and thus levels of the STAC can be initially very high, often 100 to 800 mg/L, and can then be diluted over time to a final working concentration between 20 and 160 mg/L after cell growth is initiated through the addition of a feed medium that is depleted or contains no STAC.

The fermentation liquid contained in the vessel can be inoculated with the STAC either via sterile feed or as a batch addition prior to sterilization. The inoculation is given once hydrogenase activity is determined to be impaired. The two preferred methods for determining the timing of the inoculation are: (1) the cell density level of the syngas fermentation liquid is monitored, and the syngas fermentation liquid is inoculated with the STAC when the cell density level is from 0.05 to 0.5 OD; or (2) the hydrogenase activity is monitored either via measurement of H₂ uptake or via measurement of H₂ conversion, and the syngas fermentation liquid is inoculated with the STAC when either the H₂ uptake level decreases by thirty percent or more over a 48 hour period or the H₂ conversion as a percent of mass is from zero to twenty-five percent.

The preferred STAC compound will meet the following four major criteria: (1) lack of toxicity or negative effects on acetate production, ethanol production, or cell growth at concentrations up to 1,000 mg/L; (2) exhibits a dynamic surface tension after 3000 milliseconds of incubation of less than 40 dyne/cm for seed train phase samples containing minimum cell concentration of 6.3 g DCW per liter; (3) does not adversely impact downstream processes including recycle processes (e.g., ultrafiltration or tangential flow filtration), ethanol recovery, and final product quality (i.e. absence of silicones and volatile polymers); and (4) is not metabolized into active nutrients that could support foreign growth of microorganisms, or into microbial or environmental toxins that could be activated by heating processes, or other activation processes. Given these criteria, polyol and glycol STACs were found to be preferred.

During testing based on these criteria, three polyol STACs were preferred: Cognis FBA 975US, Cognis 36K, and Cognis 153K. These preferred STACs were found to demonstrate dynamic surface tension values below 40 dyne/cm, and not adversely influence growth or ethanol production. Cognis 153K was found to show an improved kinetic mixing rate effect on dynamic surface tension, but the overall time weighted effect was not significantly different from the control.

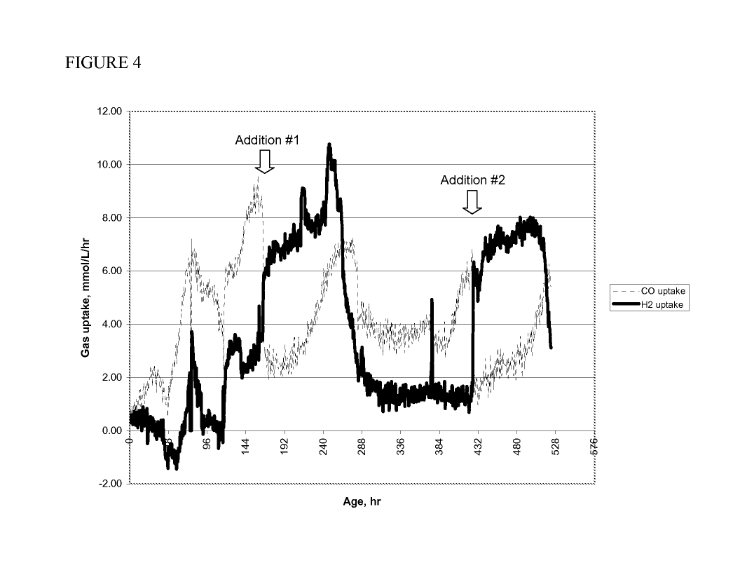

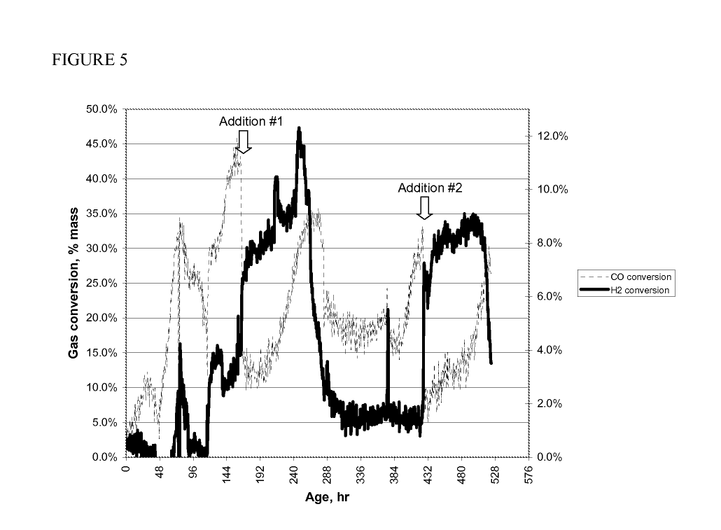

A further preferred embodiment of the present invention is to aid in recovery from a process upset during the syngas to biofuel process. After such a process upset, it is possible that the microorganisms in the fermentation liquid will be depleted, and thus it is advantageous to control CO dissolution into the fermentation liquid in order to forestall inhibition of cell growth and low gas conversion efficiencies for H₂. The STAC can be used in these circumstances to lower CO uptake and allow for increased H2 uptake, as is shown in FIG. 4 and FIG. 5.

In using the STAC to control CO dissolution during recovery from a process upset, the fermentation liquid is inoculated via sterile feed with from 10 to 400 mg per liter of fermentation liquid per day, preferably less than about 100 mg per liter per day, and the inoculation takes place when either the H₂ uptake level decreases by thirty percent or more over a 48 hour period or the H₂ conversion as a percent of mass is from zero to twenty-five percent.

Additionally, in a preferred embodiment, a PID feedback loop is maintained during steady-state fermentation based on CO and H₂ uptake rates, either manually or via PID controller, to add feed medium containing the desired concentration of STAC to a desired level, such as a level below 2.0 mg CO/linter of fermentation liquid when the cell density is greater than 0.05 but less than 0.5 OD per liter of fermentation liquid.

### Example 1

A 2 liter Sartorious Biostat B fermentor was run in CSTR mode at a gas flow rate of 0.13 vvm using a gas mix comprised of 7.5% CO, 33% H₂, 26% CO₂ and a balance of N₂. After inoculation of the reactor with Clostridium autoethanogenum, a fermentation medium described in TABLE 1 was continuously fed to the reactor at a dilution rate of 0.35±0.05 per day. After achieving a steady-state condition (9.5 days prior to inoculation), the STAC, Cognis FBA 975US was added to the fermentor via a sterile 2.0 mL pulse addition from an aqueous stock bottle. Following a single pulse addition, an immediate change in the gas uptake profile was observed as shown by FIG. 4 in which CO uptake temporarily decreased by 77%, and hydrogen uptake increased by 132%. After approximately 41 hours, a steady increase in CO uptake was observed, which was accompanied by a gradual return of the hydrogen uptake rate to baseline conditions (prior to the initial STAC pulse) by approximately 115 hours. The calculated concentration of Cognis FBA 975US following the initial pulse addition was 135 mg per liter, which was reduced to approximately 81 mg per liter at 41 hours, and to a background concentration of 20 mg/L at 115 hours.

After approximately 115 hours of operation under the standard control conditions, a second, single pulse of the STAC, Cognis FBA 975US was again added to the fermentor via a sterile 2.0 mL pulse addition from an aqueous stock bottle (135 g/L). Following a single pulse addition, an immediate change in the gas uptake profile was again observed, as shown in FIG. 4, in which CO uptake temporarily decreased by 85%, and hydrogen uptake increased by 412%. The recovery kinetics for CO and H₂ uptake followed the same behavior shown for the first addition of Cognis FBA 975US.

### Example 2

A 2 liter Sartorious Biostat B fermentor was run in CSTR mode at a gas flow rate of 0.13 vvm using a gas mix comprised of 7.5% CO, 33% H₂, 26% CO₂ and a balance of N₂. After inoculation of the reactor with Clostridium coskatii, a fermentation medium described in TABLE 1 was continuously fed to the reactor at a dilution rate of 0.31±0.05 reactor volumes per day. The STAC, Cognis FBA 975US, was added to the fermentor at a rate of 20 mg per liter per day. The agitation rate was increased in a stepwise manner to a maximum rate of 700 rpm. Each of the step increase for agitation is accompanied by an increase in gas conversion efficiency for CO; however, no increase is observed for H₂, indicating that under standard syngas fermentation conditions, H₂ uptake is not limited in its uptake by mass transfer limitations. The primary target for the STAC Cognis FBA 975US is for limiting the toxic effects of dissolved CO through mass transfer of the gas into the feed broth of the fermentation liquid.

### Example 3

Toxicity screening data was collected following a 7 day syngas fermentation of Clostridium autoethanogenum in 120 mL serum vials. The data shows the lack of adverse impacts on ethanol and cellular growth for the three STACs evaluated at a concentration of 1,000 mg/L, which included Cognis FBA 975US, Cognis 36K, and Cognis 153K.

### Example 4

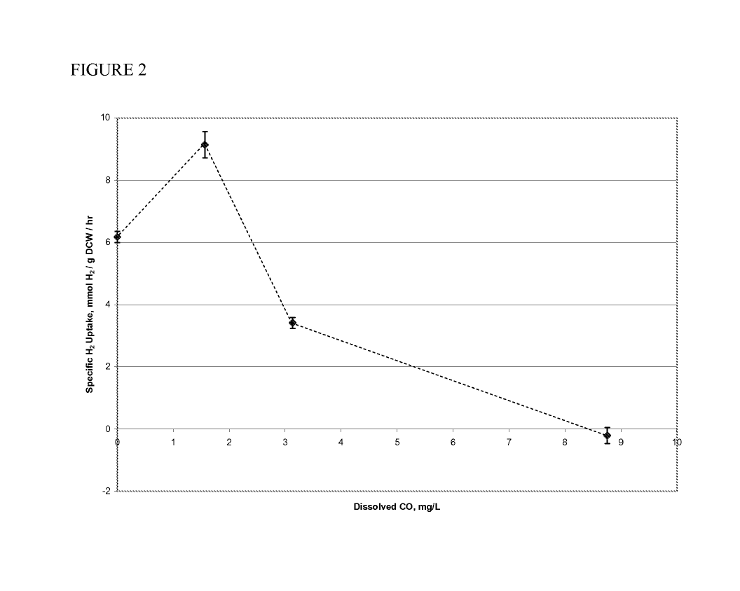

FIG. 2 gives a representative example of CO sensitivity in Clostridium ragsdalei. In FIG. 2, Continuous stirred tank reactors were fed artificial syngas blends with varying CO concentrations, using a balance of nitrogen gas. The saturated CO concentration was calculated for each condition based on CO feed gas concentration, reactor pressure, and reactor temperature at an excess gas flow condition.

TABLE 1
(a) Mineral stock solution, 40X:

| | | Molecular | |
|---|---|---|---|
| | Component and (CAS number) | formula | Amount, g |
| 1 | Sodium chloride (7647- | NaCl | 40 |
| | 14-5) | | |
| 2 | Ammonium chloride | NH₄Cl | 100 |
| | (12125-02-9) | | |
| 3 | Potassium chloride (7447- | KCl | 10 |
| | 40-7) | | |
| 4 | Potassium diphosphate | KH₂PO₄ | 20 |
| | (7778-77-0) | | |
| 5 | Magnesium sulfate, | MgSO₄•7H₂O | 20 |
| | heptahydrate (10034-99-8) | | |
| 6 | Calcium chloride, | CaCl₂•2H₂O | 4 |
| | dehydrate (10035-04-8) | | |
| 7 | Water | H₂O | 806 |

(b) Trace metal stock solution, 100X:

| | | Molecular | |
|---|---|---|---|
| | Component and (CAS number) | formula | Amount, g |
| 1 | Hydrochloric acid, 37.3% | HCl | 2.36 |
| 2 | Manganese (II) sulfate, | MnSO₄•H₂O | 3.380 |
| | monohydrate (10034-96-5) | | |
| 3 | Iron (II) sulfate, | FeSO₄•7H₂O | 12.51 |
| | heptahydrate (7782-63-0) | | |
| 4 | Cobalt (II) chloride, | CoCl₂•6H₂O | 0.357 |
| | hexahydrate (7791-13-1) | | |
| 5 | Zinc sulfate, heptahydrate | ZnSO₄•7H₂O | 2.875 |
| | (7446-20-0) | | |
| 6 | Nickel (II) chloride, | NiCl₂•6H₂O | 0.166 |
| | hexahydrate (7791-20-0) | | |
| 7 | Sodium molybdate (7631- | Na₂MoO₄ | 0.121 |
| | 95-0) | | |
| 8 | Sodium selenate (13410- | Na₂SeO₄ | 0.265 |
| | 01-0) | | |
| 9 | Water | H₂O | 978 |

(c) Vitamin stock solution, 100X:

| | | Molecular | |
|---|---|---|---|
| | Component and (CAS number) | formula | Amount, g |
| 1 | Pyridoxine, HCl (65-23-6) | C₈H₁₁NO₃•HCl | 0.060 |
| 2 | Thiamine, HCl (67-03-8) | C₁₂H₁₇N₄OS⁺ | 0.050 |
| | | Cl⁻•HCl | |
| 3 | Calcium pantothenate | C₉H₁₇NO₅ | 0.050 |
| | (137-08-6) | | |
| 4 | Nicotinic acid (59-67-6) | C₆H₅NO₂ | 0.040 |
| 5 | Biotin (58-85-5) | C₁₀H₁₆N₂O₃S | 0.018 |
| 6 | Water | H₂O | 999.8 |

(d) Cysteine stock solution, 400X:

| | | Molecular | |
|---|---|---|---|
| | Component and (CAS number) | formula | Amount, g |
| 1 | Cysteine-HCl (52-89-1) | C₃H₇NO₂S•HCl | 40 |
| 2 | Water | H₂O | 960 |

(e) Antifoam stock solution, Final use concentration is variable.

| | | Molecular | |
|---|---|---|---|
| | Component | formula | Amount, g |
| 1 | Cognis Clerol FBA975 | Proprietary | 135 |
| 2 | Water | H₂O | 865 |

What is claimed is:

1. A process for restricting carbon monoxide dissolution into a syngas fermentation liquid in a vessel during the feed phases of production of biofuels where at least 2 mg of carbon monoxide per liter of syngas fermentation liquid are present, said process comprising the steps of:
passing a feed gas stream comprising CO, H2, and CO2 through the syngas fermentation liquid,
monitoring the cell density level of the syngas fermentation liquid, and
when the cell density level is from 0.05 to 0.5 OD, inoculating the fermentation liquid with a surface tension active compound at a concentration of from 20 to 800 mg per liter of syngas fermentation liquid.

2. The process of claim 1 wherein said inoculation takes place via either sterile feed or batch addition prior to sterilization.

3. The process of claim 1, further comprising the step of diluting the fermentation liquid gradually after inoculation to a final concentration of 20 to 160 mg/L through addition of a feed medium that is depleted in or that contains no surface tension active compound.

4. The process of claim 1 wherein the surface tension active compound meets the following criteria:
lack of toxicity or negative effects on acetate, ethanol, or cell growth at concentrations up to 1000 mg/L;
exhibits a dynamic surface tension after 3000 milliseconds of incubation of less than 40 dyne/cm for broth phase samples containing a minimum cell concentration of 6.3 g DCW per liter;
does not adversely impact downstream processes including recycle processes, ethanol recovery, and final product quality; and
is not metabolized into active nutrients that could support foreign growth of microorganisms or into any environmental toxin.

5. The process of claim 1, wherein the surface tension active compound is selected from the group consisting of polyols and glycols.

6. The process of claim 1, wherein the surface tension active compound is selected from the group consisting of: Cognis FBA 975US, Cognis 36K, and Cognis 153K.

7. The process of claim 1, wherein the vessel is a stirred tank bioreactor, two-stage bioreactor, and/or bubble column bioreactor.

8. The process of claim 1, further comprising the step of passaging at least a portion of the inoculated fermentation liquid from the vessel receiving the surface tension active compound to a second vessel in a seed train, wherein said second vessel has a larger volume than the vessel receiving the surface tension active compound.

9. The process of claim 1, wherein the biofuel produced is ethanol.

10. The process of claim 1, wherein the biofuel produced is n-butanol.

11. The process of claim 1, wherein the fermentation liquid contains a microorganism selected from the group consisting of: Clostridium coskatii, Clostridium autoethanogenum, Clostridium carboxidivorans, Clostridium ljungdahlii, Butyribacterium methylotrophicum, or Clostridium ragsdalei.

12. A process for restricting carbon monoxide dissolution into a syngas fermentation liquid in a vessel during the feed phases of production of biofuels where at least 2 mg of carbon monoxide per liter of syngas fermentation liquid are present, said process comprising the steps of:
passing a feed gas stream comprising CO, H2, and CO2 through the fermentation liquid,
monitoring the level of hydrogenase activity of cells in the fermentation liquid, either via measurement of H₂ uptake in mmol/L/hour or via measurement of H₂ conversion as a percentage of mass, and
inoculating the fermentation liquid to a concentration of from 20 to 800 mg per liter of fermentation liquid with a surface tension active compound when either the H₂ uptake level decreases by thirty (30) percent or more over a 48 hour period or the H₂ conversion as a percent of mass is from zero to twenty-five (25) percent.

13. The process of claim 12, wherein said inoculation takes place via sterile feed or batch addition prior to sterilization.

14. The process of claim 12, further comprising the step of diluting the fermentation liquid gradually after inoculation to a final concentration of from 20 to 160 mg/L through addition of a feed medium that is depleted or contains no surface tension active compound.

15. The process of claim 12 wherein the surface tension active compound meets the following criteria:
lack of toxicity or negative effects on acetate, ethanol, or cell growth at concentrations up to 1000 mg/L;
exhibits a dynamic surface tension after 3000 milliseconds of incubation of less than 40 dyne/cm for broth phase samples containing a minimum cell concentration of 6.3 g DCW per liter;
does not adversely impact downstream processes including recycle processes, ethanol recovery, and final product quality; and
is not metabolized into active nutrients that could support foreign growth of microorganisms, or any environmental toxin.

16. The process of claim 12, wherein the surface tension active compound is selected from the group consisting of polyols and glycols.

17. The process of claim 12, wherein the surface tension active compound is selected from the group consisting of: Cognis FBA 975US, Cognis 36K, and Cognis 153K.

18. The process of claim 12, wherein the biofuel produced is ethanol.

19. The process of claim 12, wherein the biofuel produced is n-butanol.

20. The process of claim 12, wherein the fermentation liquid contains a microorganism selected from the group consisting of: Clostridium coskatii, Clostridium autoethanogenum, Clostridium carboxidivorans, Clostridium ljungdahlii, Butyribacterium methylotrophicum, or Clostridium ragsdalei.

21. The process of claim 12, wherein the vessel is either a stirred tank bioreactor, two-stage bioreactor, and/or bubble column bioreactor.

22. A process for restricting carbon monoxide dissolution into a syngas fermentation liquid in a vessel during recovery from a process upset where at least 2 mg of carbon monoxide per liter of fermentation liquid are present, said process comprising the steps of:
passing a feed gas stream comprising CO, H2, and CO2 through the fermentation liquid,
monitoring the level of hydrogenase activity of the cells in the fermentation liquid, either via measurement of H₂ uptake in mmol/L/hour or via measurement of H₂ conversion as a percentage of mass, and
inoculating the broth fraction of the syngas fermentation liquid with from 10 to 400 mg per liter of broth per day of a surface tension active compound when either the H₂ uptake level decreases by thirty (30) percent or more over a 48 hour period or the H₂ conversion as a percent of mass is from zero to twenty-five (25) percent.

23. The process of claim 22 wherein the inoculation takes place via sterile feed.

24. The process of claim 22, further comprising the step of maintaining a PID feedback loop based on CO and H₂ uptake rates, either manually or via PID controller, to add feed medium containing a concentration of surface tension active compound to restrict CO solubility to a desired level.

25. The process of claim 22 wherein the surface tension active compound meets the following criteria:
lack of toxicity or negative effects on acetate, ethanol, or cell growth at concentrations up to 1000 mg/L;
exhibits a dynamic surface tension after 3000 milliseconds of incubation of less than 40 dyne/cm for broth phase samples containing a minimum cell concentration of 6.3 g DCW per liter;
does not adversely impact downstream processes including recycle processes, ethanol recovery, and final product quality; and
is not metabolized into active nutrients that could support foreign growth of microorganisms, or any environmental toxin.

26. The process of claim 22, wherein the surface tension active compound is selected from the group consisting of polyols and glycols.

27. The process of claim 22, wherein the surface tension active compound is selected from the group consisting of: Cognis FBA 975US, Cognis 36K, and Cognis 153K.

28. The process of claim 22, wherein the biofuel produced is ethanol.

29. The process of claim 22, wherein the biofuel produced is n-butanol.

30. The process of claim 22, wherein the fermentation liquid contains a microorganism selected from the group consisting of: Clostridium coskatii, Clostridium autoethanogenum, Clostridium carboxidivorans, Clostridium ljungdahlii, Butyribacterium methylotrophicum, or Clostridium ragsdalei.

31. The process of claim 22, wherein the vessel is either a stirred tank bioreactor, two-stage bioreactor, and/or bubble column bioreactor.
